(19) United States
(12) Patent Application Publication
(10) Pub. No.: US 2024/0081834 A1
(43) Pub. Date: Mar. 14, 2024

(54) REVERSIBLE COMPRESSOR-DISTRACTOR FOR BONE REALIGNMENT PROCEDURES

(71) Applicant: Treace Medical Concepts, Inc., Ponte Vedra, FL (US)

(72) Inventor: Adriaan Kuyler, Ponte Vedra, FL (US)

(21) Appl. No.: 18/467,453

(22) Filed: Sep. 14, 2023

Related U.S. Application Data
(60) Provisional application No. 63/406,436, filed on Sep. 14, 2022.

(51) Int. Cl. A61B 17/15; A61B 17/17; A61B 17/64; A61B 17/66

(52) U.S. Cl. CPC A61B 17/151; A61B 17/1775; A61B 17/6425; A61B 17/66; A61B 2017/565

(57) ABSTRACT

A reversible compressor-distractor device may be used during a surgical procedure, such as a surgical procedure to correct a bunion deformity. In some examples, the reversible compressor-distractor is configured to be interchangeably used on right and left feet by reversing the orientation of the compressor-distractor. For example, the compressor-distractor may include a first engagement arm having at least two oppositely orientated pin-receiving holes on different sides of the engagement arm. The compressor-distractor may also include a second engagement arm having at least one pin-receiving hole. In use, the clinician can select an orientation of the compressor-distractor and engage pin-receiving holes associated with the selected orientation.

### CROSS-REFERENCE

This application claims the benefit of U.S. Provisional Patent Application No. 63/406,436, filed Sep. 14, 2023, the entire contents of which are incorporated herein by reference.

### TECHNICAL FIELD

This disclosure relates generally to devices and techniques for repositioning bones and, more particularly, to devices and techniques for repositioning bones in the foot.

### BACKGROUND

Bones within the human body, such as bones in the foot, may be anatomically misaligned. For example, one common type of bone deformity is hallux valgus, which is a progressive foot deformity in which the first metatarsophalangeal joint is affected and is often accompanied by significant functional disability and foot pain. The metatarsophalangeal joint is laterally deviated, resulting in an abduction of the first metatarsal while the phalanges adduct. This often leads to development of soft tissue and a bony prominence on the medial side of the foot, which is called a bunion.

Surgical intervention may be used to correct a bunion deformity. A variety of different surgical procedures exist to correct bunion deformities and may involve removing the abnormal bony enlargement on the first metatarsal and/or attempting to realign the first metatarsal relative to the adjacent metatarsal. Surgical instruments that can facilitate efficient, accurate, and reproducible clinical results are useful for practitioners performing bone realignment techniques.

### SUMMARY

In general, this disclosure is directed to devices and techniques that can be used during a surgical bone realignment procedure. In some examples, a compressor-distractor device is described that may be used during a surgical procedure, such as a surgical procedure to correct a bunion deformity. The compressor-distractor can include first and second engagement arms that each define pin-receiving holes. The first and second engagement arms can be engaged with corresponding pins inserted into underlying bone portions. The compressor-distractor may also include an actuator operatively coupled to the first and second engagement arms. For example, the first and second engagement arms of the compressor-distractor may be movably connected to each other via a threaded rod. Rotation of an actuator knob attached to the threaded rod can cause the first and second engagement arms to move relative to each other. This can apply a compression force to the bone portions in which the pins engaged with the first and second engagement arms are inserted.

In practice, a clinician performing a surgical procedure, such a bone realignment procedure on a foot of a patient, may be provided with multiple different compressor-distractor device. The clinician may be provided with one compressor-distractor configured to be used on the right foot of the patient and another compressor-distractor configured to be used on the left foot of the patient. Each compressor-distractor may be configured for the anatomical attributes of the left and right feet, respectively, such that the compressor-distractors are not interchangeably used between a surgical procedure performed on a left foot and a surgical procedure performed on a right foot. This can introduce cost and complexity to the operating environment, necessitating additional compressor-distractor inventory raising a potential that the clinician is provided with the wrong compressor-distractor for the specific surgical procedure being performed.

In accordance with some examples of the present disclosure, a reversible compressor-distractor is described that can be used in multiple different orientations. The reversible compressor-distractor may include a first engagement arm that defines multiple different pin-receiving holes and a second engagement arm that defines at least one pin-receiving hole. The pin-receiving holes provided by the first engagement arm may be angled relative to the pin-receiving hole defined by the second engagement arm. By configuring the pin-receiving holes of the first engagement arm to be angled relative to the pin-receiving hole of the second engagement arm, a correction force may be applied to one bone portion relative to another bone portion as the compressor-distractor is engaged with pins inserted into each bone portion. For example, as the compressor-distractor is guided onto pins inserted into each bone portion, the relative angulation between a pin-receiving hole defined by the first engagement arm and a pin-receiving hole defined by the second engagement arm may cause the pins, and correspondingly bone portions into which the pins are inserted, to move relative to each other. This can impart or reinforce a corrective realignment of one bone portion relative to the other bone portion.

In some configurations, the reversible compressor-distractor is configured to be flipped (e.g., inverted 180 degrees) between a first orientation and a second orientation. One or both engagement arms of the reversible compressor-distractor may include one pin-receiving hole to be used when the reversible compressor-distractor is in the first orientation and a second pin-receiving hole to be used when the reversible compressor-distractor is in the second orientation. For example, the reversible compressor-distractor may include a pin-receiving hole extending through a first side of the first engagement arm to be used when the reversible compressor-distractor is in the first orientation and another pin-receiving hole extending through a second side of the first engagement arm to be used when the reversible compressor-distractor is in the second orientation. The two pin-receiving holes may define minor image orientations with respect to each other, e.g., allowing the two pin-receiving holes to be interchangeably used as the reversible compressor-distractor is flipped between orientations.

The pin-receiving holes extending through the different sides of the first engagement arm can have entry and/or exit locations that are sufficiently offset from each other to help avoid the clinician inadvertently inserting the wrong pin-receiving hole. In some examples, the second engagement arm provides a single pin-receiving hole in which the exit of the pin-receiving hole when the reversible compressor-distractor is in the first orientation is used as the entrance of the pin-receiving hole when the reversible compressor-distractor is in the second orientation. The reversible compressor-distractor can be used in the first orientation for one foot (e.g., right or left foot) and the second orientation for the other foot.

The reversible compressor-distractor may be used during a surgical procedure in which one or more other surgical instruments are also used. For example, the compressor-distractor may be used during a procedure in which a bone preparation guide is also deployed for preparing the bones that are to be subsequent distracted and/or compressed together using the compressor-distractor. The bone preparation guide may be pinned to two different bone portions, which may be two different bones separated by a joint or two portions of the same bone (e.g., separated by a fracture or break). In either case, one end of the bone preparation guide may be pinned to one bone portion while another end of the bone preparation guide may be pinned to the other bone portion. The bone preparation guide may be pinned to the two bone portions using a pair of pins that extend parallel to each other through a pair of fixation apertures on the bone preparation guide, optionally along with one or more additional pins that may extend through one or more additional fixation apertures on the bone preparation guide that may be skewed or angled at a non-zero degree angle relative to the parallel pins. In some configurations, the bone preparation guide defines one or more slots through which a bone preparation instrument (e.g., cutting instrument) is inserted to prepare opposed end faces of the two bones.

After utilizing the bone preparation guide to prepare the two bone portions, the clinician may remove any angled pins (e.g., non-parallel pins) inserted through the bone preparation guide into the bone portions, leaving the parallel-aligned pins (e.g., a pair of parallel pins) in the bone portions. The bone preparation guide can be slide or translated along the parallel-aligned pins until the fixation apertures of the bone preparation guide come off the distal ends of the pins. At this point, the bone preparation guide may be separated from the pins, leaving the pins in the bone portions. The compressor-distractor can then be installed over the pins by threading the parallel-aligned pins through the pin-receiving holes of the compressor-distractor. For example, the clinician can select an orientation of the reversible compressor-distractor based on the foot being operated on and guide a pin inserted into one bone portion through a pin-receiving hole associated with the selected orientation. The clinician can also guide a pin inserted into the second bone portion through another pin-receiving hole of the compressor-distractor.

When the pin-receiving holes of the compressor-distractor are angled relative to each other, the process of installing the compressor-distractor on the pins will cause the pins to move. In particular, the pins may move from being substantially parallel-aligned to each other to a position in which the pins are angled relative to each other at a non-zero degree angle defined by the compressor-distractor in one or more planes, such as two or more planes. As a result, the first and second bone portions in which the pins are inserted may move a distance corresponding to the angular movement of the pins.

After installing the compressor-distractor on the pins, the clinician may actuate the actuator to move the first and second engagement arms away from each other and, as a result, move the bone portions away from each other. This can provide an enlarged separation gap between the bone portions for cleaning the inter-bone space in anticipation for fixation. For example, the clinician may remove bone chips and/or tissue debris from the inter-bone space between the two bone portions, further cut or prepare an end face of one or both bone portions, or otherwise prepare for fixation. The clinician may additionally or alternatively actuate the actuator to move the first and second engagement arms toward each other and, as a result, move the bone portions toward each other. The clinician may move the first and second engagement arms toward each other until the end faces of the bone portions are pressing against each other, resulting in a compressive force being applied to the bone portions. In some example, the clinician then fixates the bone portions by applying one or more fixation members to the bone portions.

In one example, a reversible compressor-distractor is described that includes a first engagement arm, a second engagement arm, and an actuator. The first engagement arm can include a first side pin-receiving hole for receiving a first pin inserted into a first bone portion; and a second side pin-receiving hole for receiving the first pin inserted into the first bone portion. The second side pin-receiving hole can be positioned on an opposite lengthwise side of the first engagement arm than the first side pin-receiving hole. The second engagement arm can include a pin-receiving hole for receiving a second pin inserted into a second bone portion. The actuator can be operatively coupled to the first engagement arm and the second engagement arm and configured to move the first engagement arm and the second engagement arm each other to move the first bone portion toward the second bone portion.

In another example, a method is described that includes inserting a first pin into a first bone portion and a second pin into a second bone portion. The method also includes selecting one of two different available orientations for a compressor-distractor, where the compressor-distractor includes a first engagement arm having a first side pin-receiving hole for use in a first one of the two different available orientations and a second side pin-receiving hole for use in a second one of the two different available orientations. The compressor-distractor also includes a second engagement arm having a pin-receiving hole. The method also involves inserting the compressor-distractor on the first pin by at least positioning the first pin through either the first side pin-receiving hole or the second side pin-receiving hole of the first engagement arm and inserting the second pin through the pin-receiving hole of the second engagement arm.

The details of one or more examples are set forth in the accompanying drawings and the description below. Other features, objects, and advantages will be apparent from the description and drawings, and from the claims.

### BRIEF DESCRIPTION OF THE DRAWINGS

FIGS. 1A and 1B are front views of a foot showing a normal first metatarsal position and an example frontal plane rotational misalignment position, respectively.

FIGS. 2A and 2B are top views of a foot showing a normal first metatarsal position and an example transverse plane misalignment position, respectively.

FIGS. 3A and 3B are side views of a foot showing a normal first metatarsal position and an example sagittal plane misalignment position, respectively.

FIGS. 4A and 4B are perspective views of an example reversible compressor-distractor according to disclosure.

FIGS. 5A and 5B are frontal plane side views of the example reversible compressor-distractor of FIGS. 4A and 4B.

FIGS. 6A and 6B are transverse plane side views of the example reversible compressor-distractor shown in FIGS. 5A and 5B.

FIGS. 7A and 7B are side sectional illustrations showing an example configuration of a locking mechanism.

FIGS. 8A and 8B are first and second side views of another example configuration of a reversible compressor-distractor.

FIG. 9 is a perspective view of another example configuration of a reversible compressor-distractor.

FIG. 10 is a top plan view of an example bone preparation guide that can be used with a compressor-distractor.

FIG. 11 is a perspective view of an example bone preparation guide, spacer, and tissue removing instrument check member that can be used with a compressor-distractor.

FIG. 12 is a side perspective view of a foot depicting a bone preparation instrument inserted into a joint.

FIG. 13 is a perspective view of a foot depicting a bone positioning guide on the foot prior to an alignment of a first metatarsal.

FIG. 14 is a perspective view of a foot depicting a bone positioning guide on the foot after an alignment of a first metatarsal.

FIG. 15 is a perspective view of a foot depicting a bone positioning guide on the foot after an alignment of a first metatarsal and with a spacer inserted into a joint space.

FIG. 16 is a perspective view of a foot depicting a bone preparation guide positioned on the foot.

FIG. 17 is a perspective view of a foot depicting a bone preparation guide on the foot with pins inserted through the bone preparation guide.

FIG. 18 is a perspective view of a foot depicting a removal of a bone preparation guide.

FIG. 19 is a side perspective view of a foot depicting bone plates across a joint between first and second bones.

The details of one or more examples are set forth in the accompanying drawings and the description below. Other features, objects, and advantages will be apparent from the description and drawings, and from the claims.

### DETAILED DESCRIPTION

In general, the present disclosure is directed to devices and techniques for correcting a misalignment of one or more bones. The disclosed devices and techniques can be implemented in a surgical procedure in which one bone portion is realigned relative to another bone portion. In some examples, the technique is performed on one or more bones in the foot or hand, where bones are relatively small compared to bones in other parts of the human anatomy. For example, the foregoing description generally refers to example techniques performed on the foot and, more particularly a metatarsal and cuneiform of the foot. However, the disclosed techniques may be performed on other bones. In various examples, the devices and/or techniques of the disclosure may be utilized on comparatively small bones in the foot such as a metatarsal (e.g., first, second, third, fourth, or fifth metatarsal), a cuneiform (e.g., medial, intermediate, lateral), a cuboid, a phalanx (e.g., proximal, intermediate, distal), and/or combinations thereof. The bones may be separated from each other by a tarsometatarsal (“TMT”) joint, a metatarsophalangeal (“MTP”) joint, or other joint. Accordingly, reference to a metatarsal and cuneiform herein may be replaced with other bone pairs.

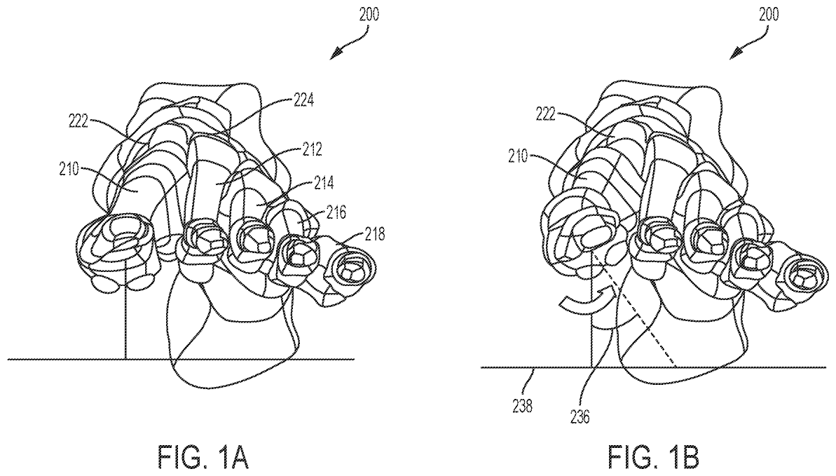

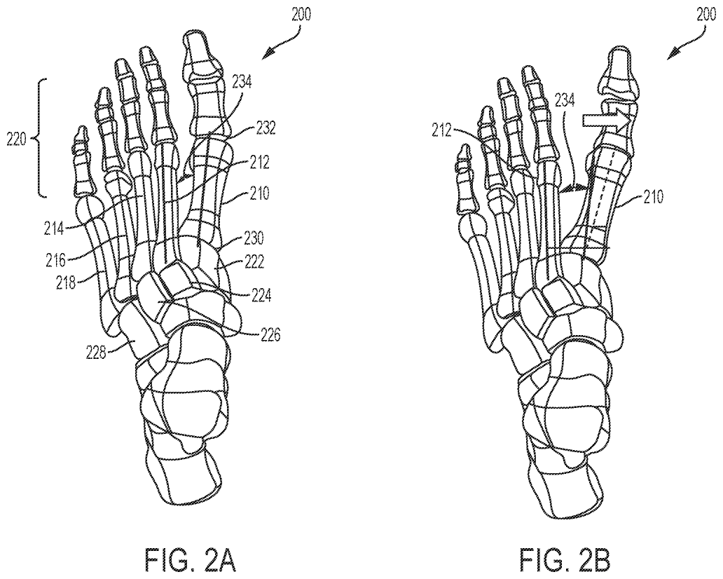

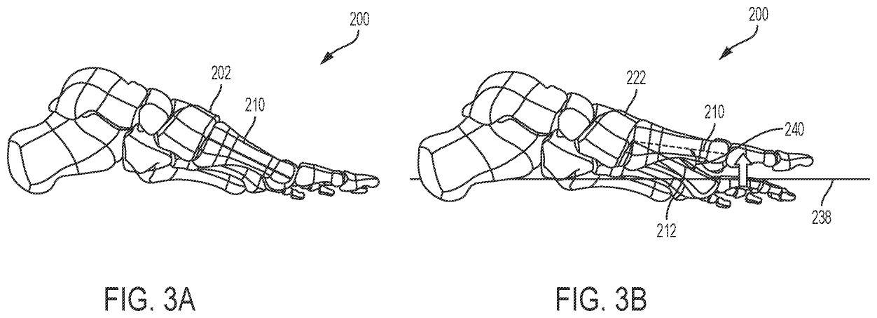

FIGS. 1-3 are different views of a foot 200 showing example anatomical misalignments that may occur and be corrected according to the present disclosure. Such misalignment may be caused by a hallux valgus (bunion), natural growth deformity, or other condition causing anatomical misalignment. FIGS. 1A and 1B are front views of foot 200 showing a normal first metatarsal position and an example frontal plane rotational misalignment position, respectively. FIGS. 2A and 2B are top views of foot 200 showing a normal first metatarsal position and an example transverse plane misalignment position, respectively. FIGS. 3A and 3B are side views of foot 200 showing a normal first metatarsal position and an example sagittal plane misalignment position, respectively. While FIGS. 1B, 2B, and 3B show each respective planar misalignment in isolation, in practice, a metatarsal may be misaligned in any two of the three planes or even all three planes. Accordingly, it should be appreciated that the depiction of a single plane misalignment in each of FIGS. 1B, 2B, and 3B is for purposes of illustration and a metatarsal may be misaligned in multiple planes that is desirably corrected.

With reference to FIGS. 1A and 2A, foot 200 is composed of multiple bones including a first metatarsal 210, a second metatarsal 212, a third metatarsal 214, a fourth metatarsal 216, and a fifth metatarsal 218. The metatarsals are connected distally to phalanges 220 and, more particularly, each to a respective proximal phalanx. The first metatarsal 210 is connected proximally to a medial cuneiform 222, while the second metatarsal 212 is connected proximally to an intermediate cuneiform 224 and the third metatarsal is connected proximally to lateral cuneiform 226. The fourth and fifth metatarsals 216, 218 are connected proximally to the cuboid bone 228. The joint 230 between a metatarsal and respective cuneiform (e.g., first metatarsal 210 and medial cuneiform 222) is referred to as the tarsometatarsal (“TMT”) joint. The joint 232 between a metatarsal and respective proximal phalanx is referred to as a metatarsophalangeal joint. The angle 234 between adjacent metatarsals (e.g., first metatarsal 210 and second metatarsal 212) is referred to as the intermetatarsal angle (“IMA”).

As noted, FIG. 1A is a frontal plane view of foot 200 showing a typical position for first metatarsal 210. The frontal plane, which is also known as the coronal plane, is generally considered any vertical plane that divides the body into anterior and posterior sections. On foot 200, the frontal plane is a plane that extends vertically and is perpendicular to an axis extending proximally to distally along the length of the foot. FIG. 1A shows first metatarsal 210 in a typical rotational position in the frontal plane. FIG. 1B shows first metatarsal 210 with a frontal plane rotational deformity characterized by a rotational angle 236 relative to ground, as indicated by line 238.

FIG. 2A is a top view of foot 200 showing a typical position of first metatarsal 210 in the transverse plane. The transverse plane, which is also known as the horizontal plane, axial plane, or transaxial plane, is considered any plane that divides the body into superior and inferior parts. On foot 200, the transverse plane is a plane that extends horizontally and is perpendicular to an axis extending dorsally to plantarly (top to bottom) across the foot. FIG. 2A shows first metatarsal 210 with a typical IMA 234 in the transverse plane. FIG. 2B shows first metatarsal 210 with a transverse plane rotational deformity characterized by a greater IMA caused by the distal end of first metatarsal 210 being pivoted medially relative to the second metatarsal 212.

FIG. 3A is a side view of foot 200 showing a typical position of first metatarsal 210 in the sagittal plane. The sagittal plane is a plane parallel to the sagittal suture which divides the body into right and left halves. On foot 200, the sagittal plane is a plane that extends vertically and is perpendicular to an axis extending proximally to distally along the length of the foot. FIG. 3A shows first metatarsal 210 with a typical rotational position in the sagittal plane. FIG. 3B shows first metatarsal 210 with a sagittal plane rotational deformity characterized by a rotational angle 240 relative to ground, as indicated by line 238.

A reversible compressor-distractor according to the disclosure can be useful during a bone positioning procedure to correct an anatomical misalignment of a bones or bones. In some applications, the reversible compressor-distractor can help establish and/or maintain a realignment between a metatarsal and an adjacent cuneiform. The metatarsal undergoing realignment may be anatomically misaligned in the frontal plane, transverse plane, and/or sagittal plane, as illustrated and discussed with respect to FIGS. 1-3 above. Accordingly, realignment may involve releasing the misaligned metatarsal for realignment and thereafter realigning the metatarsal in one or more planes, two or more planes, or all three planes. After suitably realigning the metatarsal, the metatarsal can be fixated to hold and maintain the realigned positioned.

While a metatarsal can have a variety of anatomically aligned and misaligned positions, in some examples, the term “anatomically aligned position” means that an angle of a long axis of first metatarsal 210 relative to the long axis of second metatarsal 212 is about 10 degrees or less (e.g., 9 degrees or less) in the transverse plane and/or sagittal plane. In certain embodiments, anatomical misalignment can be corrected in both the transverse plane and the frontal plane. In the transverse plane, a normal IMA 234 between first metatarsal 210 and second metatarsal 212 is less than about 9 degrees. An IMA 234 of between about 9 degrees and about 13 degrees is considered a mild misalignment of the first metatarsal and the second metatarsal. An IMA 234 of greater than about 16 degrees is considered a severe misalignment of the first metatarsal and the second metatarsal. In some embodiments, methods and/or devices according to the disclosure are utilized to anatomically align first metatarsal 210 by reducing the IMA from over 10 degrees to about 10 degrees or less (e.g., to an IMA of 9 degrees or less, or an IMA of about 1-5 degrees), including to negative angles of about −5 degrees or until interference with the second metatarsal, by positioning the first metatarsal at a different angle with respect to the second metatarsal.

With respect to the frontal plane, a normal first metatarsal will be positioned such that its crista prominence is generally perpendicular to the ground and/or its sesamoid bones are generally parallel to the ground and positioned under the metatarsal. This position can be defined as a metatarsal rotation of 0 degrees. In a misaligned first metatarsal, the metatarsal may be axially rotated between about 4 degrees to about 30 degrees or more. In some embodiments, methods and/or devices according to the disclosure are utilized to anatomically align the metatarsal by reducing the metatarsal rotation from about 4 degrees or more to less than 4 degrees (e.g., to about 0 to 2 degrees) by rotating the metatarsal with respect to the adjacent cuneiform.

A reversible compressor-distractor according to the disclosure may be useful to distract a misaligned metatarsal from an adjacent cuneiform to provide access to the end faces of the bones and/or tarsometatarsal joint. The reversible compressor-distractor may also be useful to apply a compressive force to the metatarsal and adjacent cuneiform (e.g., after preparing the end faces of the bones) to press the bones together to facilitate fixation. Additionally or alternatively, the reversible compressor-distractor may impart and/or maintain relative movement between the metatarsal and adjacent cuneiform, such as rotation and/or pivoting of one bone relative to the other bone. For example, an angular offset provided by pin-receiving holes of the reversible compressor-distractor may be effective to move the metatarsal from an anatomically misaligned position to an anatomically aligned position. As the reversible compressor-distractor is translated over pins inserted into the metatarsal and cuneiform, the angular offset of the pin-receiving holes may cause the pins to move from being generally parallel to an angular alignment dictated by the pin-receiving holes. The resulting movement of the metatarsal relative to cuneiform caused by this movement can help position the metatarsal in an aligned position.

In some examples, the reversible compressor-distractor is configured to be interchangeably used on right and left feet by reversing the orientation of the compressor-distractor. For example, the compressor-distractor may include a first engagement arm having at least two oppositely orientated pin-receiving holes on different sides of the engagement arm. The compressor-distractor may also include a second engagement arm having at least one pin-receiving hole. In use, the clinician can select an orientation of the compressor-distractor and engage pin-receiving holes associated with the selected orientation.

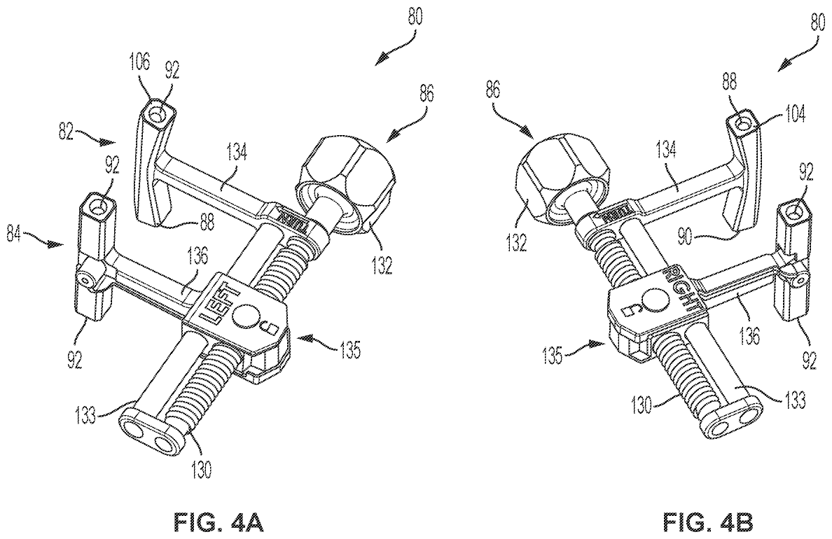

FIGS. 4A and 4B (collectively referred to as “FIG. 4”) are perspective views of an example reversible compressor-distractor 80 according to disclosure. FIG. 4A illustrates compressor-distractor 80 in a first orientation in which a first side of the compressor-distractor (labeled left side on the example device) is facing upward (e.g., plantarly) and a second side of the compressor-distractor (labeled right side on the example device) is facing downward (e.g., dorsally). FIG. 4B illustrates compressor-distractor 80 and a second orientation in which the compressor-distractor has been flipped 180° relative to the position in FIG. 4A. In particular, in FIG. 4B, compressor-distractor 80 is positioned so that side facing downward in FIG. 4A is positioned upward in FIG. 4B and, correspondingly, the side facing upward in FIG. 4A is facing downward in FIG. 4B.

In the example of FIG. 4, compressor-distractor 80 is illustrated as having a first engagement arm 82 and a second engagement arm 84. Compressor-distractor 80 also includes an actuator 86 that is operably coupled to the first engagement arm 82 and the second engagement arm 84. Actuator 86 can be actuated to move the two engagement arms toward each other and away from each other to adjust a separation distance between the two arms. As will be discussed in greater detail, each engagement arm includes at least one pin-receiving hole that is configured to receive a pin inserted into a bone. Further, the orientation of compressor-distractor 80 may be reversed from an orientation in which an end of the first engagement arm 82 and an end of the second engagement arm 84 are positioned plantarly (with the opposite end positioned dorsally) to a flipped orientation in which the end of the first engagement arm and the end of the second engagement arm are positioned dorsally (with the opposite end positioned plantarly).

For example, first engagement arm 82 may include a first side pin-receiving hole 88 on a first side of the engagement arm (FIG. 4A) and a second side pin-receiving hole 90 on a second side of the engagement arm (FIG. 4B). Second engagement arm 84 can include one or more pin-receiving holes 92 which, in the illustrated example, is shown as a single pin-receiving hole extending from the first side of compressor-distractor 80 to the second side of compressor-distractor 80. In use, a first pin can be received in the first side pin-receiving hole 88 when compressor-distractor 80 is in a first orientation or, alternatively, the first pin can be received in the second side pin-receiving hole 90 when the compressor-distractor is in a second orientation. The pin-receiving hole 92 defined by second engagement arm 84 can receive a second pin in both the first orientation and the second orientation of compressor-distractor 80.

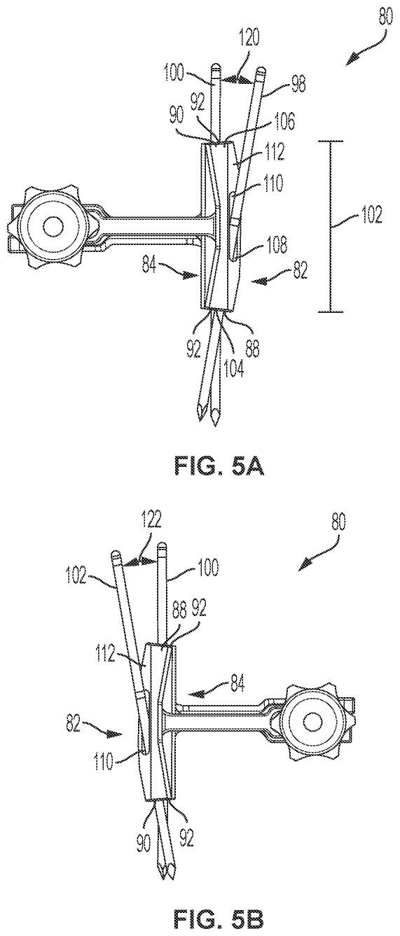

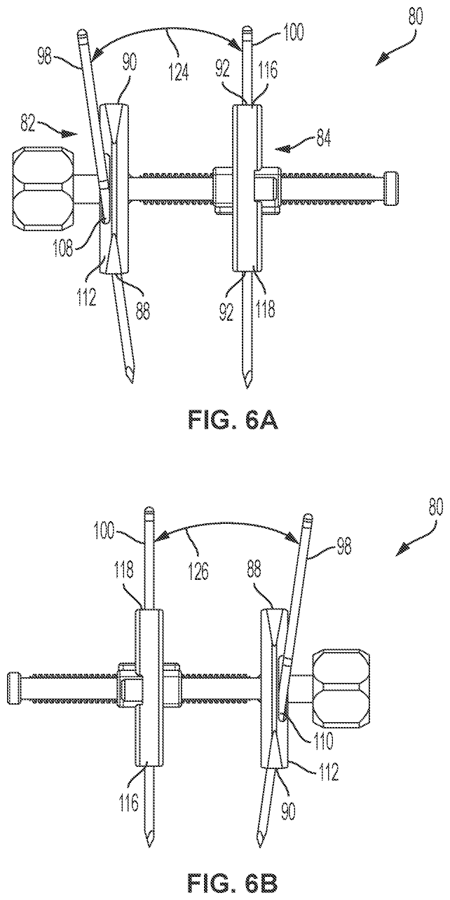

FIGS. 5A and 5B (collectively referred to as “FIG. 5”) are frontal plane side views of example configuration of compressor-distractor 80 illustrating the compressor-distractor engaged with a first pin 98 and a second pin 100. FIGS. 6A and 6B (collectively referred to as “FIG. 6”) are transverse plane side views of the example configuration of compressor-distractor 80 shown in FIGS. 5A and 5B, respectively. FIGS. 5A and 6A illustrate compressor-distractor 80 in a first orientation as shown in the perspective view of FIG. 4A. FIGS. 5B and 6B illustrate compressor-distractor 80 and a second orientation as shown in the perspective view of FIG. 4B. Compressor-distractor 80 has been flipped or rotated 180° about the length of the compressor-distractor between the example first orientation in the example second orientation illustrated in the figures.

As illustrated in the example of FIGS. 5 and 6, first side pin-receiving hole 88 and second side pin-receiving hole 90 of first engagement arm 82 can provide different pin-receiving hole options on opposite lengthwise extents of first engagement arm 82. For example, first engagement arm 82 can have a length 102 extending from a first end 104 of the first engagement arm to a second end 106 of the first engagement arm. First side pin-receiving hole 88 can be accessed from a first side (e.g., downward or dorsal side) of compressor-distractor 80, when the compressor-distractor is in a first orientation. First side pin-receiving hole 88 can provide a cavity extending at least partially along the length 102 of first engagement arm 82 that is configured to receive first pin 98, when compressor-distractor 80 is in a first orientation. Second side pin-receiving hole 90 can be accessed from a second side (e.g., downward or dorsal side) of compressor-distractor 80, when the compressor-distractor is in a second orientation. Second side pin-receiving hole 90 can also provide a cavity extending at least partially along the length 102 of first engagement arm 82 that is configured to receive first pin 98.

When compressor-distractor 80 is positioned in a first orientation (e.g., with first side pin-receiving hole 88 accessible from the bottom or dorsal side), second side pin-receiving hole 90 may be positioned on a second side (e.g., upward facing or plantar side) of compressor-distractor 80 and remain unused during the procedure. Similarly, when compressor-distractor 80 is inverted to be positioned in a second orientation (e.g., with second side pin-receiving hole 90 accessible from the bottom or dorsal side), first side pin-receiving hole 88 may be positioned on a first side (e.g., upward facing or plantar side) of compressor-distractor 80 and remain unused during the procedure. In this way, first engagement arm 82 of compressor-distractor 80 may provide at least two different pin-receiving holes accessible on different sides of the engagement arm, with the different pin-receiving holes being selectively used depending on the orientation of the compressor-distractor.

Each pin-receiving hole of compressor-distractor 80 can define an entrance opening through which an end of the pin enters the hole and an exit opening through which the end of the pin exits the hole after having traversed through a portion of the engagement arm defining the pin-receiving hole. In the illustrated arrangement, first side pin-receiving hole 88 is shown as defining an opening extending through the first end 104 of first engagement arm 82. Second side pin-receiving hole 90 is shown as defining an opening extending through the second end 106 of first engagement arm 82. The opening of first side pin-receiving hole 88 at the first end 104 of first engagement arm 82 may define a pin entry location in which first pin 98 enters the pin-receiving hole (e.g., when in the first orientation). The opening of second side pin-receiving hole 90 at the second end 106 of first engagement arm 82 may define a pin entry location in which first pin 98 enters the pin-receiving hole (e.g., when in the second orientation). Each of first side pin-receiving hole 88 and second side pin-receiving hole 90 may define a corresponding pin exit location.

For example, with reference to FIGS. 5 and 6, first side pin-receiving hole 88 of first engagement arm 82 can define a first pin exit 108. Second side pin-receiving hole 90 of first engagement arm 82 can define a second pin exit 110. First side pin-receiving hole 88 can define a linear pathway extending through first engagement arm 82 from the opening at the first end 104 of first engagement arm 82 to first pin exit 108. Second side pin-receiving hole 90 can define a linear pathway extending through first engagement arm 82 from the opening at the second end 106 of first engagement arm to second pin exit 110.

In the illustrated configuration, at least one pin-receiving hole of first engagement arm 82 extends through a sidewall of the engagement arm. For example, first engagement arm 82 can define at least one sidewall 112 forming a side surface of the engagement arm. First side pin-receiving hole 88 and/or second side pin-receiving hole 90 can extend through the sidewall 112. In the illustrated configuration, for example, first pin exit 108 of first side pin-receiving hole 88 and second pin exit 110 of second side pin-receiving hole 90 each extend through sidewall 112. First pin exit 108 and second pin exit 110 are shown extending through sidewall 112 at a same location about the perimeter of first engagement arm 82 (e.g., same radial location) but, in different examples, can be angularly offset from each other about the perimeter of the engagement arm.

Accordingly, first pin exit 108 can extend through the sidewall 112 of first engagement arm 82 at a first location, and second pin exit 110 can extend through the sidewall 112 of first engagement arm 82 at a second location. The first location can be offset along the length 102 of first engagement arm 82 from the second location. In some configurations, first side pin-receiving hole 88 does not intersect second side pin-receiving hole 90. In other configurations, first side pin-receiving hole 88 may intersect second side pin-receiving hole 90 (e.g., within the body of first engagement arm 82 or along an outer side surface of the first engagement arm beyond where each respective pin engagement hole exit sidewall 112.

For example, first side pin-receiving hole 88 can define a first longitudinal axis (e.g., shown by first pin 98 inserted through the first side pin-receiving hole in FIGS. 5A and 6A). Second side pin-receiving hole 90 can define a second longitudinal axis (e.g., shown by first pin 98 inserted through the second side pin-receiving hole in FIGS. 5B and 6B). The first longitudinal axis defined by first side pin-receiving hole 88 can intersect the second longitudinal axis defined by second side pin-receiving hole 90. Reference to a longitudinal axis is intended to refer to an axis extending along a length of a pin-receiving hole without necessitating any particular orientation relative to the length 102 of first engagement arm 82.

As briefly discussed above, second engagement arm 84 can also define a pin-receiving hole. Second engagement arm 84 may define multiple pin-receiving holes and can be configured with any of the pin-receiving configurations described herein in connection with first engagement arm 82. However, in the illustrated configuration, second engagement arm 84 is shown as having a single pin-receiving hole 92. Pin-receiving hole 92 can be oriented in a number of different ways relative to the length of second engagement arm 84. In the illustrated configuration, pin-receiving hole 92 extends parallel to (e.g., co-linear with) the length of second engagement arm 84.

For example, as seen in FIG. 6, second engagement arm 84 can have a length 114 extending from a first end 116 to a second end 118. The pin-receiving hole defined by second engagement arm 84 can extend through the first end 116 of the second engagement arm and through the second end 118 of the engagement arm. When compressor-distractor 80 is used in the first orientation, an opening at the first end 116 of second engagement arm 84 can define a pin entry location in which an end of second pin 100 enters the second engagement arm. In this orientation, an opening at the second end 118 of second engagement arm 84 can define a pin exit where the end of second pin 100 exits the second engagement arm. By contrast, when compressor-distractor 80 is used in the second orientation, the opening at the second end 118 of second engagement arm 84 can define the pin entry location, and the opening at the first end 116 of the second engagement arm can define the pin exit location.

During use, a selected one of first side pin-receiving hole 88 and second side pin-receiving hole 90 of first pin engagement arm 82 can receive first pin 98. In addition, the pin-receiving hole 92 defined by second engagement arm 84 can receive second pin 100. The first pin 98 and second pin 100 can be inserted into different bones or bone portions being worked upon. In the case of a bone realignment procedure, for example, first pin 98 can be inserted into a metatarsal (e.g., first metatarsal 210) and second pin 100 can be inserted into a cuneiform (e.g., medial cuneiform 222). The pin-receiving holes can anchor compressor-distractor 80 to the bones being compressed and/or distracted via the pins inserted through the holes and into the underlying bones. Additionally or alternatively, the pin-receiving holes can be used to impart relative movement between one bone in which first pin 98 is inserted and another bone in which second pin 100 is inserted.

For example, first side pin-receiving hole 88 and second side pin-receiving hole 90 may each be angled relative to one or more pin-receiving holes 92 defined by second engagement arm 84. The pin-receiving holes can be angled relative to each other at a non-zero degree angle such that, when compressor-distractor 80 is inserted over first and second pins 98, 100 (which may be a substantially parallel set of pins), the angled receiving holes causes the pins to move relative to each other to align with the pin-receiving holes. The direction and extent of movement imposed by the angled pin-receiving holes of compressor-distractor 80 may vary depending on the desired surgical application in which the compressor-distractor is being used. In the case of a misaligned metatarsal, such as a bunion procedure for instance, the pin-receiving holes may be angled to impart a frontal plane rotation and/or a sagittal plane translation. As a result, when compressor-distractor 80 is installed over pins position in the metatarsal and adjacent cuneiform, the angled pin-receiving holes may cause the metatarsal to rotate in the frontal plane relative to the cuneiform and/or translate in the sagittal plane (e.g., downwardly or plantarly) to help correct a misalignment of the metatarsal.

As discussed above, first side pin-receiving hole 88 can define a first longitudinal axis and second side pin-receiving hole 90 can define a second longitudinal axis. In addition, the pin-receiving hole 92 through second engagement arm 84 can define a third longitudinal axis (e.g., shown by second pin 100 inserted through the pin-receiving hole in FIGS. 5 and 6). The first longitudinal axis defined by first side pin-receiving hole 88 and/or the second longitudinal axis defined by second side pin-receiving hole 90 can be angled relative to the third longitudinal axis defined by pin-receiving hole 92 and one or more planes, such as in two or more planes. For example, the first longitudinal axis defined by first side pin-receiving hole 88 can be angled relative to the third longitudinal axis defined by pin-receiving hole 92 in one or both of a frontal plane and a sagittal plane. The second longitudinal axis defined by second side pin-receiving hole 90 can be angled relative to the third longitudinal axis defined by pin-receiving hole 92 in one or both of the frontal plane and the sagittal plane. In some configurations, either the first longitudinal axis or the second longitudinal axis is not angled relative to the third longitudinal axis but, instead, is parallel to the third longitudinal axis.

FIG. 5 illustrates example frontal plane angular offsets between the first longitudinal axis defined by first side pin-receiving hole 88 and the third longitudinal axis defined by pin-receiving hole 92 as well as between the second longitudinal axis defined by second side pin-receiving hole 90 and the third longitudinal axis defined by pin-receiving hole 92. In this example, the first longitudinal axis defined by first side pin-receiving hole 88 is angled relative to the third longitudinal axis defined by pin-receiving hole 92 in the frontal plane by an angle 120. The second longitudinal axis defined by second side pin-receiving hole 90 is angled relative to the third longitudinal axis defined by pin-receiving hole 92 in the frontal plane by an angle 122. While angles 120 and 122 may vary, in some examples, each angle may range from 2° to 20°, such as from 6° to 15°, or from 8° to 12°, or approximately 10°. The respective longitudinal axes may be offset in a direction that causes the metatarsal to rotate laterally as the compressor-distractor 80 is installed over first and second pins 98, 100. For example, when compressor-distractor 80 is positioned on the medial side of the foot, the relative frontal plane angulation between the pin-receiving holes may cause first pin 98 to rotate toward the lateral side of the foot relative to second pin 100.

In addition to or in lieu of providing a fontal plane angulation, compressor-distractor 80 may be configured to impart sagittal plane rotation, when the compressor-distractor 80 is installed over first and second pins 98, 100. For example, when installed over first and second pins 98, 100 positioned in the metatarsal and cuneiform (e.g., substantially parallel pins), the relative angulation of the pin-receiving holes may cause the metatarsal to rotate or flex plantarly (e.g., such that the distal end of the metatarsal is rotated plantarly about the TMT joint).

FIG. 6 illustrates example sagittal plane offsets between the first longitudinal axis defined by first side pin-receiving hole 88 and the third longitudinal axis defined by pin-receiving hole 92 as well as between the second longitudinal axis defined by second side pin-receiving hole 90 and the third longitudinal axis defined by pin-receiving hole 92. In this example, the first longitudinal axis defined by first side pin-receiving hole 88 is angled relative to the third longitudinal axis defined by pin-receiving hole 92 in the sagittal plane by an angle 124. The second longitudinal axis defined by second side pin-receiving hole 90 is angled relative to the third longitudinal axis defined by pin-receiving hole 92 in the sagittal plane by an angle 126. While angles 124, 126 may vary, in some examples, each angle may range from 2° to 12°, such as from 5° to 9°, or from 6° to 8°, or approximately 7°. The pin-receiving holes may be offset relative to each other in a direction that causes the metatarsal to rotate (e.g., downwardly or plantarly) in the sagittal plane as the compressor-distractor 80 is installed over first and second pins 98, 100.

First side pin-receiving hole 88 may define a same relative angle or angles, 120, 124 relative to pin-receiving hole 92 as the angle or angles 122, 124 defined between the second side pin-receiving hole 90 in pin-receiving hole 92. For example, one or more angles defined between the first longitudinal axis and the third longitudinal axis may be equal and opposite to one or more corresponding angles defined between the second longitudinal axis and the third longitudinal axis. When so configured, first side pin-receiving hole 88 and second side pin-receiving hole 90 may define a mirrored relationship with respect to each other. In other words, the angular relationship between first side pin-receiving hole 88 and pin-receiving hole 92 may be the same as the angular relationship between second side pin-receiving hole 90 in pin-receiving hole 92. This can configure first side pin-receiving hole 88 and second side pin-receiving hole 90 to provide the same angular relationship with pin-receiving hole 92 when compressor-distractor 80 is a reverse between the first and second orientations. In other configurations, however, first side pin-receiving hole 88 may have one or more different angles relative to pin-receiving hole 92 in the one or more corresponding angles defined between second side pin-receiving hole 90 in pin-receiving hole 92.

In general, features described as pin-receiving holes may be void spaces extending through a portion of compressor-distractor 80 and configured (e.g., sized and/or shaped) to pass a pin inserted into a bone therethrough. While the pin-receiving holes may have any polygonal (e.g., square, rectangle) or arcuate (e.g., curved, elliptical) shape, the pin-receiving holes may typically have a circular cross-sectional shape. In some examples, the pin-receiving holes have a diameter ranging from 0.1 mm to 10 mm, such as from 0.5 mm to 4 mm. The pin-receiving holes may have a length (e.g., extending through the thickness of first engagement arm 82 or second engagement arm 84) ranging from 5 mm to 50 mm, such as from 10 mm to 25 mm.

As briefly discussed above in connection with FIG. 4, compressor-distractor 80 can open and close to compress and distract the bones to which to the compressor-distractor is secured. To facilitate movement, compressor-distractor 80 is illustrated as having an actuator 86. Actuator 86 is configured to control movement of first engagement arm 82 relative to second engagement arm 84. Actuator 86 may be implemented using any feature that provides controllable relative movement between the two engagement arms, such as rotary movement, sliding movement, or other relative translation. In some configurations, actuator 86 is configured to move first and second engagement arms 82, 84 at least 1 mm away from each other, such as a distance ranging from 1 mm to 45 mm, a distance ranging from 1 mm to 5 mm, or a distance ranging from 1 mm to 2.5 mm during distraction. Actuator 86 may be actuated during compression until the faces of the bones to which compressor-distractor 80 is attached are suitably compressed and/or the sidewall faces of first and second engagement arms 82, 84 contact each other.

In the example of FIG. 4, actuator 86 is illustrated as including a shaft 130 operatively connected to the first engagement arm 82 and the second engagement arm 84. Shaft 130 may be threaded and actuator 86 may further include a knob 132 coupled to the shaft. Rotation of knob 132 in one direction may cause first engagement arm 82 to move closer to second engagement arm 84, while rotation of the knob in the opposite direction can cause the first engagement arm to move away from the second engagement arm.

First engagement arm 82 and/or second engagement arm 84 may be directly connected to shaft 130 or may be indirectly connected via one or more intermediate portions. For example, as illustrated, compressor-distractor 80 includes a first bridging arm 134 between actuator 86 and first engagement arm 82, particularly between shaft 130 connected to actuator 86 and first engagement arm 82. Compressor-distractor 80 also includes a second bridging arm 136 between actuator 86 and second engagement arm 84, particularly between shaft 130 connected to actuator 86 and second engagement arm 84. As illustrated, first bridging arm 134 and second bridging arm 136 extend generally orthogonally from a lengthwise extent of first engagement arm 82 and second engagement arm 84, respectively. For example, first bridging arm 134 and second bridging arm 136 may be connected to first engagement arm 82 and second engagement arm 84, respectively, at approximately a midpoint along the length of each engagement arm. This can help allow compressor-distractor 80 to be flipped between orientations and used interchangeably between different orientations.

To secure actuator 86 to compressor-distractor 80, the actuator may be fixedly connected to one of the engagement arms. For example, shaft 130 of actuator 86 may be fixedly attached along its length to first engagement arm 82 and rotatable relative to the second engagement arm 84. As a result, when knob 132 is rotated, second engagement arm 84 may move along the length of shaft 130 towards and/or away from first engagement arm 82. This provides relative movement between the two arms while first engagement arm 82 remains in a fixed position relative to actuator 86.

In some examples, the mechanical advantage provided by actuator 86 can be controlled to help limit peak torque and prevent overtightening (over compression) of the bone portions connected to compressor-distractor 80. The mechanical advantage can be controlled by controlling the geometry of knob 132, the thread lead of shaft 130, and/or drag (e.g., mechanical losses from friction). In some examples, the thread lead of shaft 130 is greater than 1.5 mm per turn, such as greater than 2.0 mm per turn, or greater than 2.5 mm per turn, such as from 2.0 to 3.0 mm per turn. Accordingly, peak torque input from an average user may be less than 30 pounds of force, such as less than 25 pounds of force.

To help stabilize first engagement arm 82 relative to second engagement arm 84 during movement along shaft 130, compressor-distractor 80 may also include one or more unthreaded shafts extending parallel to the threaded shaft. In FIG. 4, for example, actuator 86 has an unthreaded shaft 133 extending parallel to threaded shaft 130 and helps stabilize second engagement arm 84 as it moves along the threaded shaft towards and away from first engagement arm 82.

During actuation of actuator 86, compressor-distractor 80 may have a tendency to move up first pin 98 and/or second pin 100. To help secure compressor-distractor 80 to one or both pins, the compressor-distractor may include one or locking mechanisms. The locking mechanism can interface with a pin engaged with compressor-distractor to lock the compressor-distractor to pin (e.g., at a particular sagittal plane position). In FIG. 4, compressor-distractor 80 is illustrated as including a locking mechanism 135.

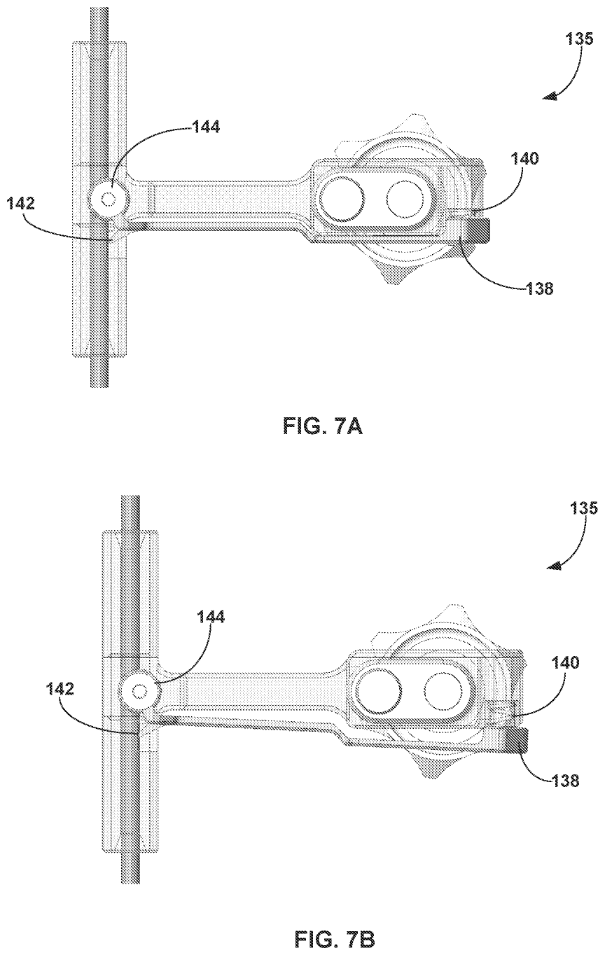

FIGS. 7A and 7B are side sectional illustrations showing an example configuration of locking mechanism 135. FIG. 7A illustrates locking mechanism 135 in an unlocked position. FIG. 7B illustrates locking mechanism 135 in a locked position. In the illustrated example, locking mechanism 135 includes a locking arm 138 operatively connected to a spring 140. Locking arm 138 has a contact end 142 configured to press against first pin 98 or second pin 100. Locking arm 138 can rotate about a pivot connection 144. A center of rotation of pivot connection 144 may be aligned with an edge of the pin being locked. Contact end 142 of locking arm 138 may provide an orthogonal moment to the pin being locked, e.g., when spring 140 is extended in the locking position. For example, the force applied by contact end 142 may be applied approximately 90 degrees relative to the length of the pin being locked. This can provide bi-directional locking, e.g., resisting relative movement between the pin and contact end 142 independent of whether compressor-distractor 80 is used in the first orientation or the second orientation, which may bias the compressor-distractor along the length of the pin in opposite directions depending on the orientation of the compressor-distractor.

Compressor-distractor 80 may be fabricated from any suitable material or combination of materials, such as metal (e.g., stainless steel) and/or polymeric materials. In some configurations, compressor-distractor 80 is fabricated from a radiolucent material such that it is relatively penetrable by X-rays and other forms of radiation, such as thermoplastics and carbon-fiber materials. Such materials are useful for not obstructing visualization of bones using an imaging device when the bone positioning guide is positioned on bones.

While compressor-distractor 80 has generally been described as having a first engagement arm 82 having first and second side pin-receiving holes angled in one or more planes relative to an orthogonal pin-receiving hole 92 defined by second engagement arm 84 (e.g., to receive second pin 100 extending orthogonally in the dorsal-to-plantar direction), other configurations of pin-receiving holes can be used. In general, relative angles between sets of pin-receiving holes defined by first engagement arm 82 and second engagement arm 84 may be achieved by relatively angling the pin-receiving holes of both arms.

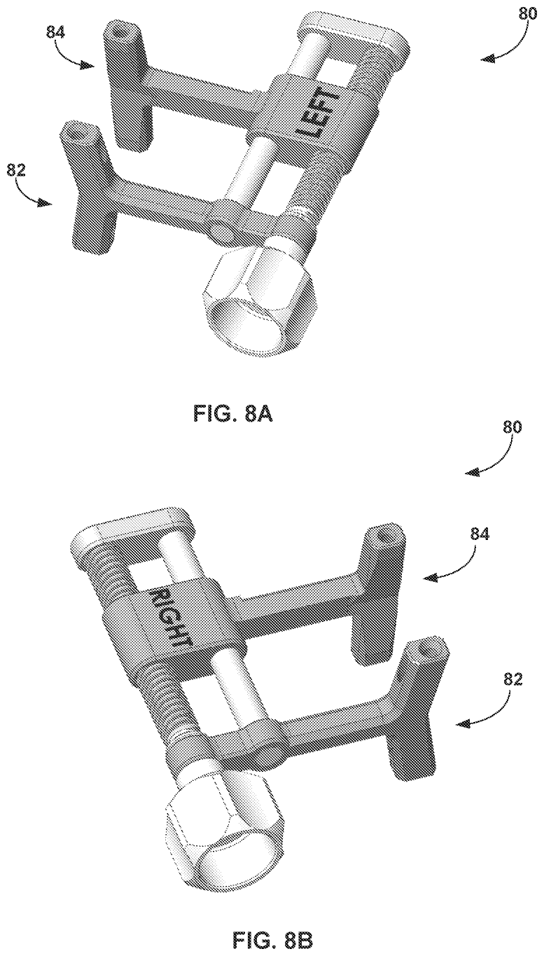

For example, instead of pin-receiving hole 92 of second engagement arm 84 extending through the first and second ends of the second engagement arm, the pin-receiving hole may be angled and extend out through a sidewall of the second engagement arm. When so configured, second engagement arm may also include a first side pin-receiving hole (e.g., optionally with a first pin exit hole through a sidewall of the engagement arm) and a second side pin-receiving hole (e.g., optionally with a second pin exit hole through a sidewall of the engagement arm). FIGS. 8A and 8B are first and second side views of another example configuration of compressor-distractor 80 in which the second engagement arm does not include an orthogonal pin-receiving hole extending through a length of the engagement arm but instead includes off-axis pin-receiving holes.

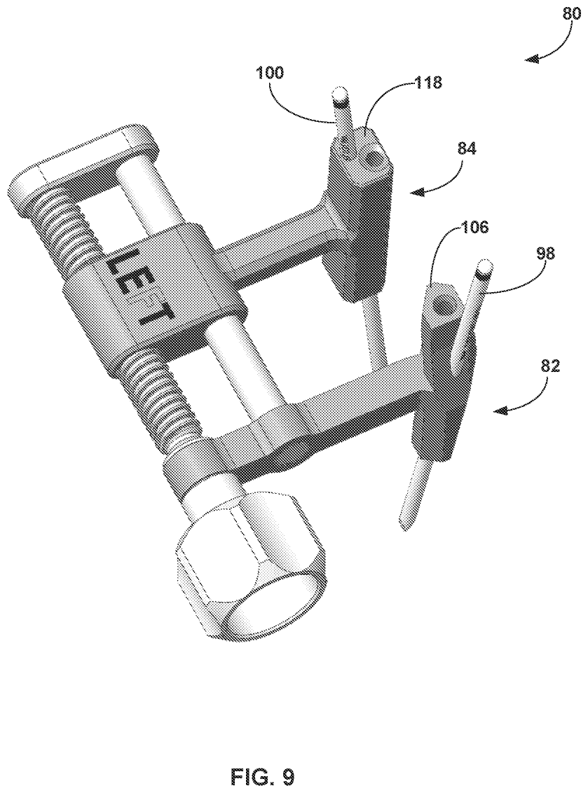

In addition, while compressor-distractor 80 has generally been described as having a first engagement arm 82 having first and second side pin-receiving holes with pin exit locations extending through a sidewall of the engagement arm, in other configurations, one or both of the first and second side pin-receiving holes may have a pin entrance on one end of the engagement arm and a pin exit on another end of the engagement arm. This may position the pin entrance for a pin-receiving hole to be used in one orientation of compressor-distractor 80 adjacent to the pin exit for a pin-receiving hole to be used in a second orientation of the compressor-distractor. FIG. 9 is a perspective view of another example configuration of compressor-distractor 80 in which the first and second side pin-receiving holes have an adjacent pin entrance and pin exit through an end of the engagement arm.

A compressor-distractor according to the disclosure may be used as part of a surgical procedure in which at least two pins are inserted into different bones or different portions of the same bone. The at least two pins may be inserted in generally parallel alignment and/or the pins may be realigned during the surgical procedure so as to be substantially parallel (e.g., prior to installation of compressor-distractor 80). The two pins may be substantially parallel in that the pins are positioned side-by-side and have substantially the same distance continuously between the two pins in each of the three planes (e.g., the distance varies by less than 10%, such as less than 5% across the lengths of the pins in any given plane, with different continuous distances in different planes). Compressor-distractor 80 can be inserted over the parallel pins by threading the parallel pins into the pin-receiving holes of the device, thereby causing the pins to move from a substantially parallel alignment to an angled alignment dictated by the angulation of the pin-receiving holes. Compressor-distractor 80 may then be used to distract the bone portions into which the pins are inserted (e.g., by actuating actuator 86 to draw the bone portions away from each other) and/or compress the bone portions into which the pins are inserted (e.g., by actuating actuator 86 to move the bone portions towards each other).

In some examples, compressor-distractor 80 is used as part of a metatarsal realignment procedure in which a metatarsal is realigned relative to an adjacent cuneiform and/or metatarsal in one or more planes, such as two or three planes. Additional details on example bone realignment techniques and devices with which compressor-distractor 80 may be used are described in U.S. Pat. No. 9,622,805, titled “BONE POSITIONING AND PREPARING GUIDE SYSTEMS AND METHODS,” filed on Dec. 28, 2015 and issued Apr. 18, 2017, and U.S. Pat. No. 9,936,994, titled “BONE POSITIONING GUIDE,” filed on Jul. 14, 2016 and issued on Apr. 10, 2018; US Patent Publication No. 2017/0042599 titled “TARSAL-METATARSAL JOINT PROCEDURE UTILIZING FULCRUM,” filed on Aug. 14, 2016; and US Provisional Patent Application No. (attorney docket no. 71212.61.USP1) titled “MINIMALLY INVASIVE METATARSAL REALIGNMENT,” filed concurrently herewith. The entire contents of each of these documents are hereby incorporated by reference.

The pins over which compressor-distractor 80 is installed may be used to pin and/or guide another medical instrument used during the surgical technique. For example first and second pins 98, 100 may be used to pin a first medical instrument to the bones or bone portions being operated upon. The medical instrument can be removed over the parallel pins, leaving the pins inserted into the bone or bone portions, and compressor-distractor 80 subsequently placed over the pins.

For example, in the case of a metatarsal realignment procedure, first and second pins 98, 100 may be used to pin a bone preparation guide to a foot being operated upon. The bone preparation guide can be used to prepare an end face of a metatarsal and an adjacent end face of a corresponding cuneiform. The bone preparation guide can be taken off the first and second pins and compressor-distractor 80 installed on the pins.

For example, the clinician can select one of two different available orientations in which to use compressor-distractor 80 (e.g., with the first side positioned plantarly or the second side positioned plantarly). The clinician can select the desired orientation by flipping the compressor-distractor (e.g., 180 degrees about the length of the device) from the first one of the two different available orientations to the second one of the two different available orientations. One orientation may correspond to a procedure performed on a right foot of the patient while the other orientation may correspond to a procedure performed on a left foot of the patient.

With the desired orientation of compressor-distractor 80 selected, the clinician can insert the compressor-distractor on the first and second pins 98, 100 by aligning pin entrances associated with the selected orientation of the compressor-distractor with the ends of the pins inserted into bones. The clinician can advance compressor-distractor 80 down on the pins, e.g., until the ends of the pins project out of corresponding pin exits. In some examples, the clinician advance compressor-distractor 80 down on the pins until the ends of the first and send engagement arms contact underlying skin and/or bone. The relatively large, substantially planar end faces of the engagement arms may minimize soft tissue damage when the first and second pins 98, 100 are inserted percutaneously and the ends of the engagement arms contact underlying skin. In either case, the relative angulation of the pin-receiving holes defined by the arms may cause the first and second pins 98, 100 (and, correspondingly, bone portions in which the pins are inserted) to move relative to each other. With compressor-distractor 80 positioned on first and second pins 98, 100, the compressor-distractor can be manipulated to open the joint space between the metatarsal and cuneiform, e.g., to facilitate joint cleanup, and/or manipulated to compress the two bones together for fixation.

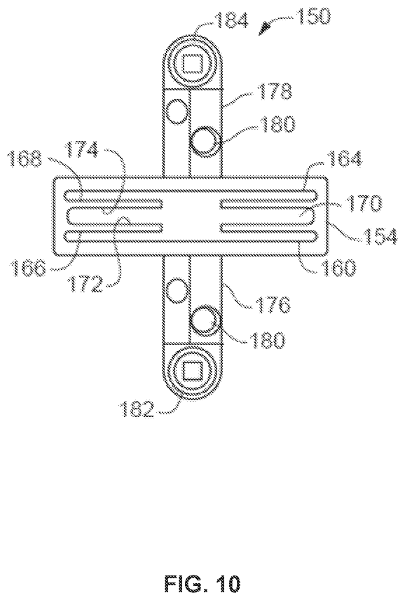

FIG. 10 illustrates an example bone preparation guide 150 that may be used as part of a surgical procedure involving compressor-distractor 80. In some examples, bone preparation guide 150 includes a body 154 defining a first guide surface 160 to define a first preparing plane and a second guide surface 164 to define a second preparing plane. A tissue removing instrument (e.g., a saw, rotary bur, osteotome, etc., not shown) can be aligned with the surfaces to remove tissue (e.g., remove cartilage or bone and/or make cuts to bone). The first and second guide surfaces 160, 164 can be spaced from each other by a distance, (e.g., between about 2 millimeters and about 10 millimeters, such as between about 4 and about 7 millimeters). In the embodiment shown, the first and second guide surfaces are parallel, such that cuts to adjacent bones using the guide surfaces will be generally parallel.

In some configurations, as shown in FIG. 10, a first facing surface 166 is positioned adjacent the first guide surface 160 and/or a second facing surface 168 is positioned adjacent the second guide surface 164. In such configurations, the distance between the first guide surface and the first facing surface defines a first guide slot, and the distance between the second guide surface and the second facing surface defines a second guide slot. Each slot can be sized to receive a tissue removing instrument to prepare the bone ends. The first and second slots may be parallel or skewed. In the illustrated example, the facing surfaces each contain a gap, such that the surface is not a single, continuous surface. In other embodiments, the facing surfaces can be a single, continuous surface lacking any such gap.

An opening 170 can be defined by the body 154 between the first and second guide surfaces. The opening can be an area between the guide surfaces useful for allowing a practitioner to have a visual path to bones during bone preparation and/or to receive instruments. In the configuration shown, the opening extends across the body and a distance from a surface 172 opposite of the first facing surface 166 to a surface 174 opposite of the second facing surface 168.

The illustrated bone preparation guide also includes a first end 176 extending from the body 154 in a first direction and a second end 178 extending from the body in a second direction. The second direction can be different than the first direction (e.g., an opposite direction). As shown, each of the first end and the second end can include at least one fixation aperture 180 configured to receive a fixation pin to secure the bone preparation guide to an underlying bone. For example, first end 176 of bone preparation guide 150 may define a first fixation aperture through which first pin 98 (FIG. 5) is inserted and the second end 178 of bone preparation guide 150 may define a second fixation aperture through which second pin 100 (FIG. 5) is inserted. These two fixation apertures may or may not be parallel aligned, such that first and second pins 98, 100 extend through the holes parallel to each other. The first end 176 and/or the second end 178 of bone preparation guide 150 may also defined one or more additional fixation apertures that are angled (at a non-zero degree angle) or otherwise skewed relative to the two parallel fixation apertures.

In use, a clinician may insert the two pins (e.g., parallel pins) through fixation apertures 180 and may optionally insert one or more angled pins through the one or more angled fixation apertures. This combination of parallel and angled pins may prevent bone preparation guide 150 from being removed from the underlying bones being worked upon. When the clinician has completed using the bone preparation guide, the angled pin or pins may be removed leaving the two parallel pins inserted into the underlying bones. Bone preparation guide 150 can be slid or otherwise moved up and off the parallel pins and compressor-distractor 80 thereafter inserted down over the pins.

In some examples as shown in FIG. 10, bone preparation guide 150 can also include a first adjustable stabilization member 182 engaged with the first end 176 and/or a second adjustable stabilization member 184 engaged with the second end 178. Each of the members can be threaded and engage a threaded aperture defined by the ends. The elevation of each end can be adjusted with respect to a bone by adjusting the stabilization member. In some embodiments, as shown, the stabilization members are cannulated such that they can receive a fixation pin.

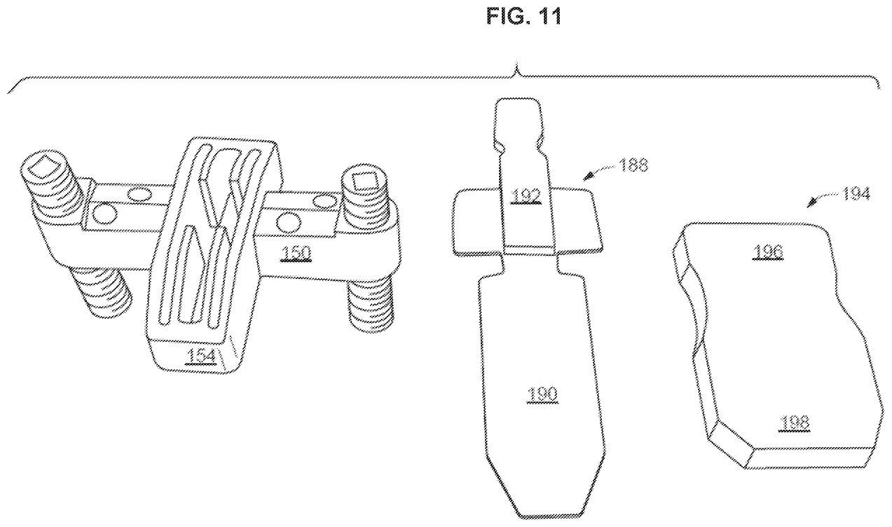

With reference to FIG. 11, bone preparation guide 150 may include or be used with a spacer 188 that extends downward from the body 154. Spacer 188 may be configured to be placed into a joint (e.g., within the TMT joint). In some embodiments, the spacer 188 is selectively engageable with the body of the bone preparation guide and removable therefrom. The spacer can have a first portion 190 configured to extend into a joint space and a second portion 192 engageable with the body 154. In the embodiment shown, the spacer can be received within opening 170, such that the spacer extends from the body in between the first and second guide surfaces. Such a spacer can be useful for positioning the body at a desired position with respect to a joint and for properly positioning the guide with respect to bones to be cut in more than one plane (e.g., three planes selected from more than one of a frontal plane, a transverse plane, and a sagittal plane). The distance between the spacer and the first guide surface can define a length of tissue removal (e.g., bone or cartilage to be cut) from a first bone, and the distance between the spacer and the second guide surface can define a length of tissue removal (e.g., bone or cartilage to be cut) from a second bone.

As also shown in FIG. 11, bone preparation guide 150 may include or be used with a tissue removal location check member 194. Tissue removal check member 194 may be engageable with the body 154 and configured to extend to a first bone and a second bone. The tissue removal location check member can have a first portion 196 configured to extend into contact with first and second bones and a second portion 198 engageable with the body. In the embodiment shown, the tissue removal check member 194 is configured to extend in the body 154 at both the first and second guiding surfaces. The tissue removal location check member 194 may be useful for allowing a practitioner to see where a tissue removing instrument guided by the surfaces will contact the bone to be prepared.

Bone preparation facilitated by bone preparation guide 150 can be useful, for instance, to facilitate contact between leading edges of adjacent bones, separated by a joint, or different portions of a single bone, separated by a fracture, such as in a bone alignment and/or fusion procedure. A bone may be prepared using one or more bone preparation techniques. In some applications, a bone is prepared by cutting the bone. The bone may be cut transversely to establish a new bone end facing an opposing bone portion. Additionally or alternatively, the bone may be prepared by morselizing an end of the bone. The bone end can be morselized using any suitable tool, such as a rotary bur, osteotome, or drill. The bone end may be morselized by masticating, fenestrating, crushing, pulping, and/or breaking the bone end into smaller bits to facilitate deformable contact with an opposing bone portion.

During a surgical technique utilizing compressor-distractor 80, a bone may be moved from an anatomically misaligned position to an anatomically aligned position with respect to another bone. Further, both the end of the moved bone and the facing end of an adjacent end may be prepared for fixation. In some applications, the end of at least one of the moved bone and/or the other bone is prepared after moving the bone into the aligned position. In other applications, the end of at least one of the moved bone and/or the other bone is prepared before moving the bone into the aligned position.

Movement of one bone relative to another bone can be accomplished using one or more instruments and/or techniques. In some examples, bone movement is accomplished using a bone positioning device that applies a force to one bone at a single location, such that the bone both translates and rotates in response to the force. This may be accomplished, for example, using a bone positioning device that engages and/or applies a force between a bone to be moved (e.g., a metatarsal) and another bone (e.g. an anchoring bone, such as another metatarsal or a cuneiform). In some examples, the bone positioning device includes a bone engagement member, a tip, a mechanism to urge the bone engagement member and the tip towards each other, and an actuator to actuate the mechanism. Additionally or alternatively, bone movement may be accomplished using compressor-distractor 80 by imparting movement to one bone relative to another bone as the compressor-distractor is positioned on the pins, causing the pins to move out of their alignment and resulting in movement of the underlying bones in one plane (e.g., frontal plane, sagittal plane, transverse plane), two or more planes, or all three planes. As yet a further addition or alternative, a clinician may facilitate movement by physically grasping a bone, either through direct contact with the bone or indirectly (e.g., by inserting a K-wire, grasping with a tenaculum, or the like), and moving his hand to move the bone.

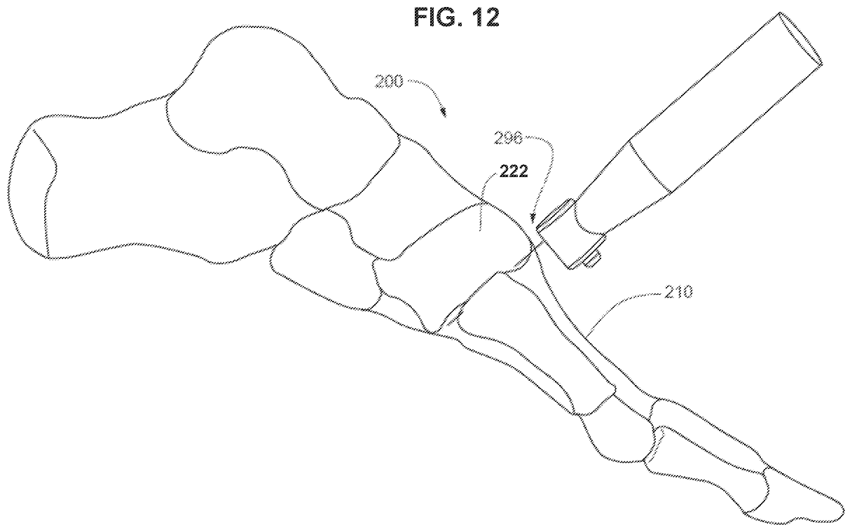

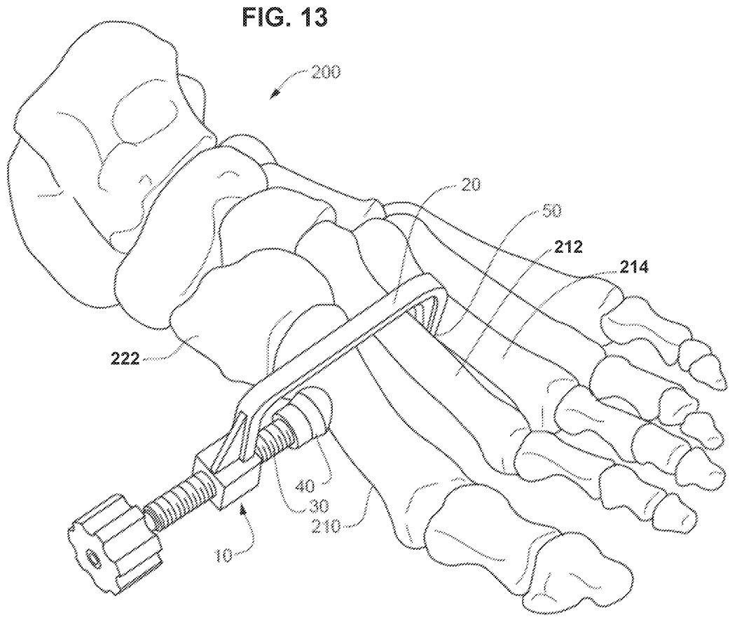

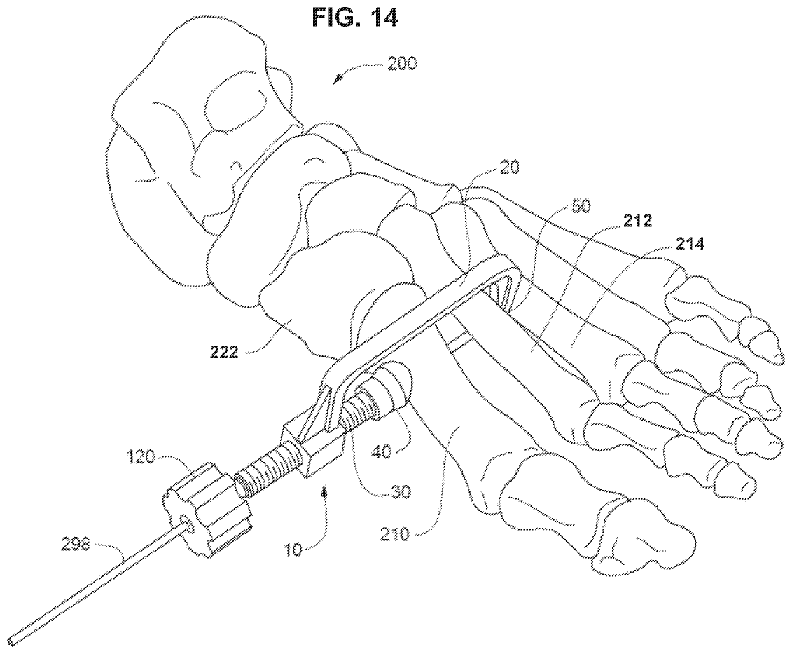

An example method for preforming a bone alignment procedure utilizing a compressor-distractor according to the disclosure will now be described with respect to FIGS. 12-19 depicting a foot 200 having a first metatarsal 210, a medial cuneiform 222, and a second metatarsal 212. Unless otherwise indicated, the example steps described can be carried out in any order and need not be performed in the order described.

After customary surgical preparation and access, a bone preparation instrument 296 can be inserted into the joint (e.g., first tarsal-metatarsal joint) to release soft tissues and/or excise the plantar flare from the base of the first metatarsal 210, as shown in FIG. 12. Excising the plantar flare may involve cutting plantar flare off the first metatarsal 210 so the face of the first metatarsal is generally planar. This step helps to mobilize the joint to facilitate a deformity correction. In some embodiments, the dorsal-lateral flare of the first metatarsal may also be excised to create space for the deformity correction (e.g., with respect to rotation of the first metatarsal). In certain embodiments, a portion of the metatarsal base facing the medial cuneiform can be removed during this mobilizing step.

An incision can be made and, if a bone positioning instrument is going to be used, a tip 50 of a bone positioning guide 10 inserted on the lateral side of a metatarsal other than the first metatarsal 210, such as the second metatarsal 212. As shown in FIG. 10, the tip can be positioned proximally at a base of the second metatarsal 212 and a third metatarsal 214 interface. A surface of a bone engagement member 40 can be placed on the proximal portion of the first metatarsal 210. In some embodiments, the bone engagement member engages a medial ridge of the first metatarsal 210. As shown, the body 20 of the positioning guide can be generally perpendicular to the long axis of the second metatarsal 212.

In applications utilizing bone positioning guide 10, the actuator on the bone positioning guide can be actuated to reduce the angle (transverse plane angle between the first metatarsal and the second metatarsal) and rotate the first metatarsal about its axis (frontal plane axial rotation). The first metatarsal 210 can be properly positioned with respect to the medial cuneiform 222 by moving the bone engagement member 40 bone positioning guide with respect to the tip 50 of the bone positioning guide. In some embodiments, such movement simultaneously pivots the first metatarsal with respect to the cuneiform and rotates the first metatarsal about its longitudinal axis into an anatomically correct position to correct a transverse plane deformity and a frontal plane deformity. Again, however, other applications utilizing compressor-distractor 80 may be performed without utilizing bone positioning guide 10 and/or utilizing a bone positioning guide of different design.

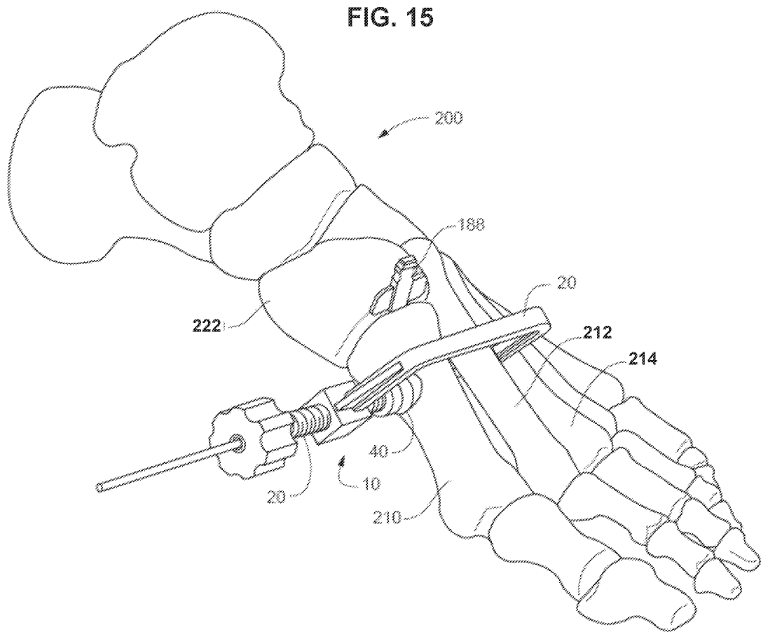

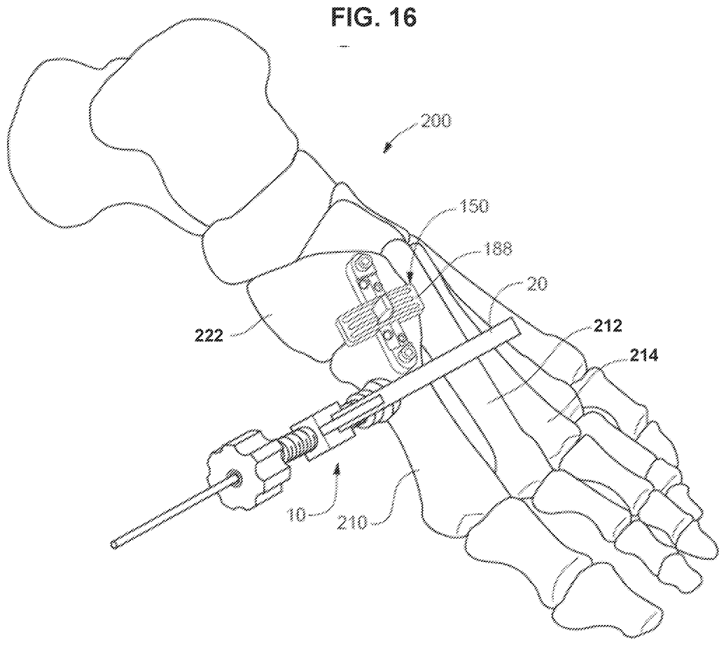

Independent of whether bone positioning guide 10 is used, an example technique may include positioning joint spacer 188 can be positioned within the joint between the first metatarsal and the medial cuneiform, as illustrated in FIG. 15. Bone preparation guide 150 can be placed over the joint spacer 188 as shown in FIG. 16 and engaged with the joint spacer to set a position and orientation of the bone preparation guide relative to the joint. In other embodiments, bone preparation guide 150 is placed on the bones without using joint spacer 188 to aid with positioning.

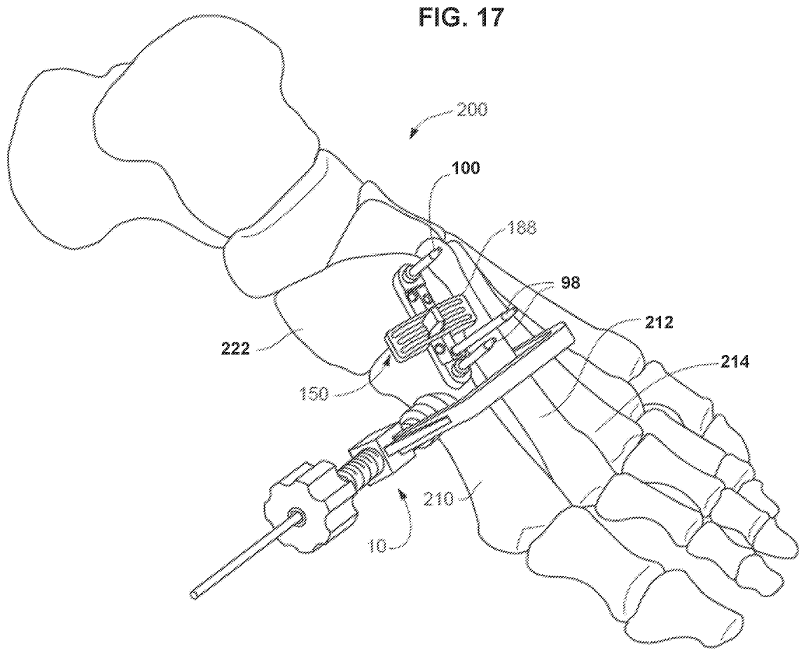

As depicted in FIG. 17, one or more fixation pins can be inserted into apertures of the bone preparation guide 150 to secure the guide to the first metatarsal 210 and the medial cuneiform 222. The fixation pins inserted into the apertures of bone preparation guide 150 includes a first fixation pin 98 and second fixation pin 100. The first and second fixation pins 98, 100 may be inserted in substantially parallel alignment. The first and second fixation pins 98, 100 may project at least 25 mm above the surface of the bones into which the pins are inserted, such as at least 50 mm, or at least 75 mm. One or more additional pins can be inserted at an angle or in a converging orientation to help prevent movement of the bone preparation guide 150 during a tissue removing step. After insertion of the pins, the spacer 188 (if used) can optionally be removed in embodiments having a selectively engageable spacer.

In some applications, the end of the first metatarsal 210 facing the medial cuneiform 222 can be prepared with a tissue removing instrument 296 guided by a guide surface of bone preparation guide 150 (e.g., inserted through a slot defined by a first guide surface and a first facing surface). In some embodiments, the first metatarsal 210 end preparation is done after at least partially aligning the bones, e.g., by actuating bone positioning guide 10 before preparing the end of first metatarsal 210. In other embodiments, the first metatarsal 210 end preparation is done before the alignment of the bones, e.g., by preparing the end of the first metatarsal 210 before installing compressor-distractor 80 to cause realignment.

In addition to preparing the end of first metatarsal 210, the end of the medial cuneiform 222 facing the first metatarsal 210 can be prepared with the tissue removing instrument 296 guided by a guide surface of bone preparation guide 150 (e.g., inserted through a slot defined by a second guide surface and a second facing surface). In some embodiments, the medial cuneiform 222 end preparation is done after the alignment of the bones. In yet other embodiments, the medial cuneiform 222 end preparation is done before the alignment of the bones. In embodiments that include cutting bone or cartilage, the cuneiform cut and the metatarsal cut can be parallel, conforming cuts. In some examples, a saw blade can be inserted through a first slot to cut a portion of the medial cuneiform and the saw blade can be inserted through a second slot to cut a portion of the first metatarsal.

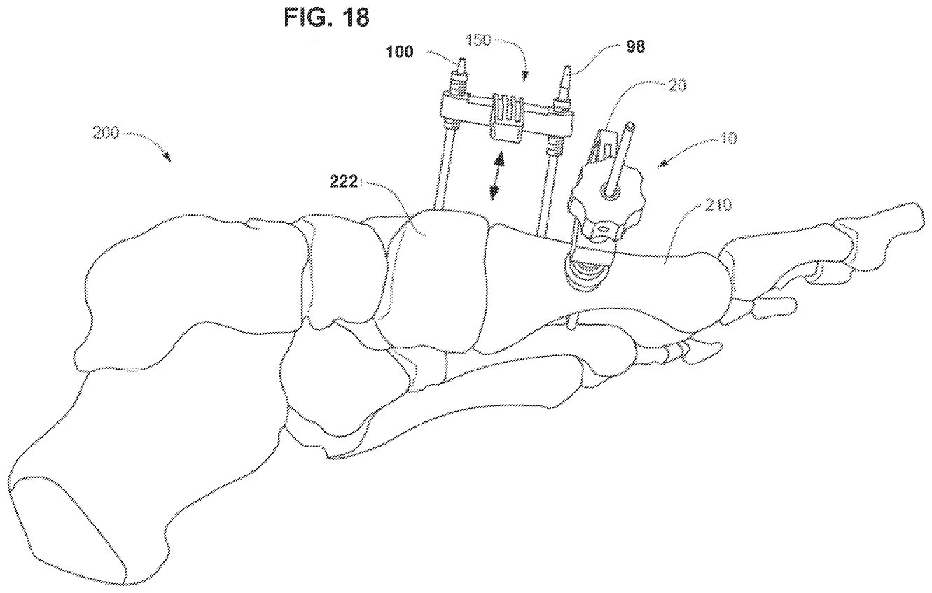

Any angled/converging pins can be removed and the bone preparation guide 150 can be lifted off the first and second pins 98, 100, as shown in FIG. 18. These pins, which may or may not be substantially parallel pins, can receive compressor-distractor 80. For example, the clinician can align entrance locations of selected pin-receiving holes with the pins and then slide compressor-distractor 80 down over the pins toward the underlying bones and/or skin covering the bones. The clinician may adjust the spacing between first engagement arm 82 and second engagement arm 84 by actuating actuator 86 until the separation distance corresponds to the spacing between first and second pins 98, 100, before installing the compressor-distractor down over the pins. In the process of inserting compressor-distractor 80 on the pins, the angulation between the pin-receiving holes can cause first and second pins 98, 100 to shift to an alignment corresponding to the angular position of the pin-receiving holes. As the first and second pins 98, 100 move relative to each other, first metatarsal 210 may move relative to medial cuneiform 222 a direction and distance corresponding to the movement of first pin 98 relative to second pin 100.

In applications where bone positioning guide 10 is utilized, the bone positioning guide may be removed before or after bone preparation guide 150 is removed and compressor-distractor 80 is installed. In either case, in some examples, a temporary fixation device such as an olive pin, k-wire, or other fixation structure may be used to maintain the position of the underlying bones (e.g., first metatarsal 210 relative to medial cuneiform 222) while bone preparation guide 150 is removed and compressor-distractor 80 is installed.

With compressor-distractor 80 pinned to underlying bones (e.g., first metatarsal 210 and medial cuneiform 222), actuator 86 may be actuated to distract the underlying bones. For example, the clinician may turn knob 132 to cause second engagement arm 84 to move away from first engagement arm 82, opening or enlarging a gap between the underlying bones. When pinned to first metatarsal 210 and medial cuneiform 222, the clinician can actuate actuator 86 to open the TMT joint.

With the underlying bones distracted, the clinician may clean or otherwise prepare the space between the bones and/or the end face of one or both bones. The clinician may clean the space by removing excess cartilage, bone, and/or other cellular debris that may natively exist or may have been created during the bone preparation step that may inhibit infusion.

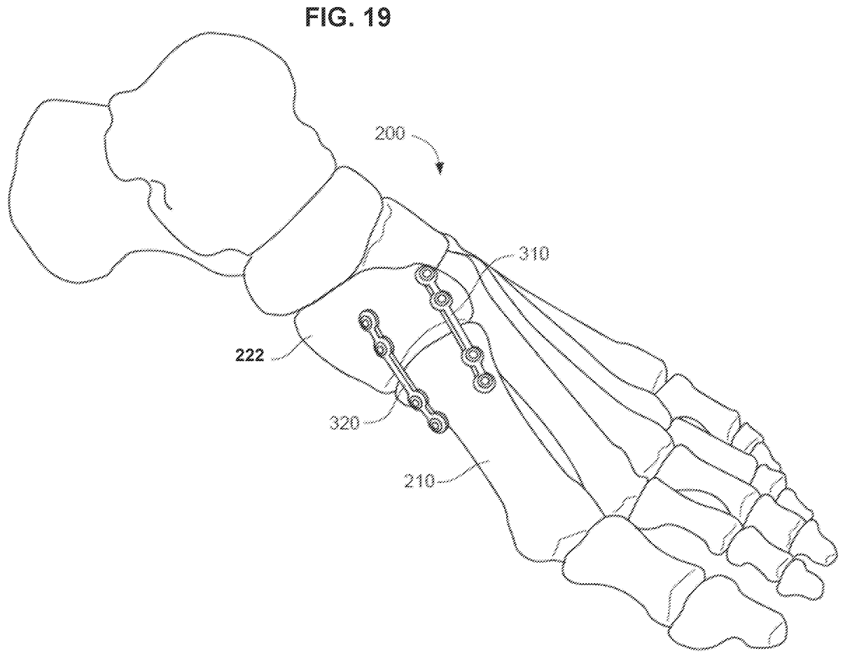

Independent of whether the clinician utilizes compressor-distractor 80 to distract the underlying bones for cleaning, the clinician may actuate actuator 86 to compress the bones together for permanent fixation infusion. The clinician may turn knob 132 to cause second engagement arm 84 to move toward first engagement arm 82, for example until the end faces of the underlying bones contact each other and/or a compressive force is applied through pins 98, 100 to the end faces. With the two end faces pressed together via compressor-distractor 80, the clinician may provisionally or permanently fixate the bones or bones portions together. For example, one or more bone fixation devices can be applied across the joint and to the two bones to stabilize the joint for fusion, such as two bone plates positioned in different planes. FIG. 19 illustrates an example fixation device arrangement that includes a first bone plate 310 positioned on a dorsal-medial side of the first metatarsal and medial cuneiform and a second bone plate 320 positioned on a medial-plantar side of the first metatarsal and the medial cuneiform. In other embodiments, one or more staples or other implants can be used in addition to or in lieu of a plate.

Compressor-distractor devices and techniques have been described. In some examples, a compressor-distractor according to disclosure is included in a disposable, sterile kit that includes associated surgical instrumentation, such as bone positioning guide and/or a preparation guide described herein. Other components that may be included within the sterile kit include bone fixation devices, bone fixation screws, pins for insertion into pin-receiving holes, and the like.

Various examples have been described. These and other examples are within the scope of the following claims.

1. A reversible compressor-distractor comprising:
(a) a first engagement arm comprising:
(i) a first side pin-receiving hole for receiving a first pin inserted into a first bone portion; and
(ii) a second side pin-receiving hole for receiving the first pin inserted into the first bone portion, wherein the second side pin-receiving hole is on an opposite lengthwise side of the first engagement arm than the first side pin-receiving hole;
(b) a second engagement arm comprising a pin-receiving hole for receiving a second pin inserted into a second bone portion; and
(c) an actuator operatively coupled to the first engagement arm and the second engagement arm, the actuator being configured to move the first engagement arm and the second engagement arm each other to move the first bone portion toward the second bone portion.

2. The reversible compressor-distractor of claim 1, wherein:
the first engagement arm has a length extending from a first end to a second end;
the first side pin-receiving hole extends through the first end of the first engagement arm; and
the second side pin-receiving hole extends through the second end of the first engagement arm.

3. The reversible compressor-distractor of claim 2, wherein:
the first engagement arm has a sidewall;
the first side pin-receiving hole extends from the first end of the first engagement arm through the sidewall at a first location; and
the second side pin-receiving hole extends from the second end of the first engagement arm through the sidewall at a second location.

4. The reversible compressor-distractor of claim 3, wherein the first location is offset along the length of the first engagement arm from the second location.

5. The reversible compressor-distractor of claim 2, wherein a longitudinal axis defined by the first side pin-receiving hole intersects a longitudinal axis defined by the second side pin-receiving hole at a location between the first end and the second end of the first engagement arm.

6. The reversible compressor-distractor of claim 3, wherein:
the first side pin-receiving hole defines a pin entry location at the first location of the sidewall and a pin exit location at the first end of the first engagement arm; and
the second side pin-receiving hole defines the pin entry location at the second location of the sidewall and the pin exit location at the second end of the first engagement arm.

7. The reversible compressor-distractor of claim 1, wherein the reversible compressor-distractor is configured to be flipped from a first orientation in which a first end is positioned closer to the first bone portion than a second end and the first pin is inserted through the first side pin-receiving hole to a second orientation in which the second end is positioned closer to the first bone portion than the first end and the first pin is inserted through the second side pin-receiving hole.

8. The reversible compressor-distractor of claim 1, wherein:
the second engagement arm has a length extending from a first end to a second end; and
the pin-receiving hole extends from the first end through the second end of the second engagement arm.

9. The reversible compressor-distractor of claim 1, wherein:
the first side pin-receiving hole defines a first longitudinal axis;
the second side pin-receiving hole defines a second longitudinal axis;
the pin-receiving hole of the second engagement arm defines a third longitudinal axis; and
the first longitudinal axis and the second longitudinal axis are each angled relative to the third longitudinal axis.

10. The reversible compressor-distractor of claim 9, wherein an angle between the first longitudinal axis and the third longitudinal axis is equal and opposite to an angle between the second longitudinal axis and the third longitudinal axis.

11. The reversible compressor-distractor of claim 9, wherein:
the first longitudinal axis is angled relative to the third longitudinal axis in one or both of a frontal plane and a sagittal plane; and
the second longitudinal axis is angled relative to the third longitudinal axis in one or both of the frontal plane and the sagittal plane.

12. The reversible compressor-distractor of claim 11, wherein the first longitudinal axis and the second longitudinal axis are each angled relative to the third longitudinal axis in the frontal plane at an angle ranging from 2 degrees to 20 degrees.

13. The reversible compressor-distractor of claim 11, wherein the first longitudinal axis and the second longitudinal axis are each angled relative to the third longitudinal axis in the sagittal plane at an angle ranging from 5 degrees to 12 degrees.

14. The reversible compressor-distractor of claim 1, wherein the actuator comprises a threaded shaft operatively connected to the first engagement arm and the second engagement arm and at least one unthreaded shaft extending parallel to the threaded shaft.

15. The reversible compressor-distractor of claim 1, further comprising a first bridging arm between the actuator and the first engagement arm and a second bridging arm between the actuator and the second engagement arm.

16. The reversible compressor-distractor of claim 1, further comprising a locking mechanism configured to lock a position of one or both of the first pin and the second pin, when inserted into the reversible compressor-distractor.

17. The reversible compressor-distractor of claim 16, wherein the locking mechanism comprise a locking arm operatively connected to a spring, and the locking arm has a contact end configured to press against one of the first pin or the second pin.

18. The reversible compressor-distractor of claim 17, wherein the locking arm is configured to apply an orthogonal moment to one of the first pin or the second pin.

19. A method comprising:
inserting a first pin into a first bone portion and a second pin into a second bone portion;
selecting one of two different available orientations for a compressor-distractor, wherein the compressor-distractor includes a first engagement arm having a first side pin-receiving hole for use in a first one of the two different available orientations and a second side pin-receiving hole for use in a second one of the two different available orientations, the compressor-distractor further including a second engagement arm having a pin-receiving hole; and
inserting the compressor-distractor on the first pin by at least positioning the first pin through either the first side pin-receiving hole or the second side pin-receiving hole of the first engagement arm and inserting the second pin through the pin-receiving hole of the second engagement arm.

20. The method of claim 19, wherein selecting one of two different available orientations for the compressor-distractor comprises flipping the compressor-distractor from the first one of the two different available orientations to the second one of the two different available orientations.

21. The method of claim 19, wherein:
the first engagement arm has a length extending from a first end to a second end;
the first side pin-receiving hole extends through the first end of the first engagement arm;
the second side pin-receiving hole extends through the second end of the first engagement arm; and
inserting the compressor-distractor on the first pin by at least positioning the first pin through either the first side pin-receiving hole or the second side pin-receiving hole of the first engagement arm comprise inserting the first pin into either the first end or the second end of the compressor-distractor.

22. The method of claim 21, wherein:
the first engagement arm has a sidewall;
the first side pin-receiving hole extends from the first end of the first engagement arm through the sidewall at a first location;
the second side pin-receiving hole extends from the second end of the first engagement arm through the sidewall at a second location; and
inserting the compressor-distractor on the first pin by at least positioning the first pin through either the first side pin-receiving hole or the second side pin-receiving hole comprises causing the first pin to exit from either the sidewall at the first location or the second location.

23. The method of claim 19, wherein a longitudinal axis defined by the first side pin-receiving hole and a longitudinal axis defined by the second side pin-receiving hole are each angled relative to a longitudinal axis defined by the pin-receiving hole of the second engagement arm, and inserting the compressor-distractor on the first pin and the second pin comprises causing the first bone portion to move relative to the second bone portion.

24. The method of claim 19, further comprising:
prior inserting the first pin and the second pin, positioning a first fixation aperture of a bone preparation guide over the first bone portion and a second fixation aperture of the bone preparation guide over the second bone portion, the first and second fixation apertures being parallel to each other,
wherein inserting the first pin and the second pin comprises inserting the first pin through the first fixation aperture into the first bone portion and the second pin through the second fixation aperture into the second bone portion.

25. The method of claim 24, further comprising, prior to inserting the compressor-distractor on the first pin and on the second pin, sliding the bone preparation guide off the first pin and the second pin while leaving the first pin inserted into the first bone portion and the second pin inserted into the second bone portion.

26. The method of claim 19, further comprising actuating the actuator on the compressor-distractor to cause the first engagement arm to move toward the second engagement arm and, in turn, cause the first bone portion to move toward the second bone portion.

27. The method of claim 26, further comprising fixating the first bone portion to the second bone portion while the first engagement arm is moved toward the second engagement arm and causing the first bone portion to compress against the second bone portion.

28. The method of claim 19, wherein the first bone portion is a metatarsal and the second bone portion is a cuneiform.
